(19) United States
(12) Patent Application Publication
(10) Pub. No.: US 2025/0295830 A1
(43) Pub. Date: Sep. 25, 2025

(54) HEMOSTATIC SPONGE COMPRISING GELATIN AND CHITOSAN

(71) Applicant: THE AMERICAN UNIVERSITY IN CAIRO, New Cairo (EG)

(72) Inventors: Hassan Mohamed El-Said AZZAZY, New Cairo (EG); Wesam Awad SARHAN, New Cairo (EG); Mousa ElSayed EISayed SALEM, Alexandria (EG)

(73) Assignee: THE AMERICAN UNIVERSITY IN CAIRO, New Cairo (EG)

(21) Appl. No.: 18/834,810

(22) PCT Filed: Feb. 6, 2023
(86) PCT No.: PCT/US2023/012419

§ 371 Date: Jul. 31, 2024

Related U.S. Application Data
(60) Provisional application No. 63/307,411, filed on Feb. 7, 2022.

(51) Int. Cl. A61L 26/00

(52) U.S. Cl. CPC A61L 26/0085; A61L 26/0023; A61L 26/0038; A61L 2400/04

(57) ABSTRACT

A hemostatic sponge comprising crosslinked gelatin and/or gelatin/chitosan, a method for manufacturing it, and methods of its use.

### CROSS-REFERENCE TO RELATED APPLICATION

This application claims priority to U.S. Provisional Application No. 63/307,411, filed Feb. 7, 2022, which is incorporated by reference for all purposes.

### BACKGROUND OF THE INVENTION

### Field of the Invention

The present invention relates to the field of medicine and specifically to hemostatic sponges which contain gelatin or gelatin/chitosan, to a method of producing these sponges, and to their uses for hemostasis.

### Description of Related Art

Excessive bleeding complicates healing processes and results in a high risk of morbidity. For example, bleeding during surgery limits the ability of the surgeon to operate efficiently and post-operative bleeding inhibits healing and recovery.

Since early reports of gelatin-based hemostatic scaffolds by Correll in 1945, different forms of cross-linked gelatin have been utilized to stop bleeding by absorbing blood and by expanding to compress the wound. However, cross-linked gelatin sponges suffer from very low wettability which necessitates either adding a wetting agent within the gelatin sponge or pre-wetting sponges before application. This low wettability has required prehydration of crosslinked gelatin sponges prior to application to a wound in order to attain effective hemostatic activity, rapid clotting action, and complete fluid absorption. This poor wettability is reflected in the instructions for use of commercially available sponges such as CUTANPLAST®). GELITA-SPON® and COLTENE® which require a sponge be prehydrated, for example, by immersion in sterile isotonic saline.

Moreover, prehydration is also often required to uniformly hydrate a sponge so that it has uniform hemostatic properties over its surface area. Insufficient or improper hydration can cause distortions in shape or irregular zones of hydration or dryness in the sponge and cause unanticipated difficulties in placing the sponge or fitting it to a wound. This prehydration step is troublesome, time consuming, and potentially life threatening given the limited time frame for controlling hemorrhages especially in critical surgical procedures.

Chitosan-based dressings are widely utilized as they exhibit good hemostatic ability. Among the commonly utilized chitosan-based sponges are CLO-SUR® (hypertext transfer protocol://scionbiomed.com/topical-hemostasis/; last accessed Sep. 22, 2021) and HEMCON® chitosan dressings (hypertext transfer protocol secure://tricolbiomedical.com/; last accessed Feb. 1, 2022), which offer good hemostatic ability and wettability, however both of them suffer from a strong acidic odor and are configured and utilized as sheets instead of as three dimensional sponge structures. This sheet structure reduces their hemostatic ability for treating inter-cavity bleeding and deep wounds.

No chitosan/gelatin sponges are currently present in the market. Prior attempts to make such a sponge have been unsuccessful because they produced sponges with poor hemostatic ability characterized by a low absorption of blood of only about 25% of their weight and produced sponges containing deleterious or toxic amounts of crosslinkers and other extraneous compounds not required for hemostasis. Additionally, as mentioned above, current available gelatin sponges suffer from very low wettability thus necessitating a pre-wetting step.

In view of the above, the inventors sought to develop a hemostatic sponge that can be quickly used without prehydration delay and which does not require the incorporation of any or significant amounts of ingredients such as wetting agents, surfactants, or blowing agents which complicate the manufacturing process and which can expose a patient to extraneous chemicals including those which can interfere with clotting and wound healing. The inventors developed a method that allows production of gelatin and gelatin/chitosan sponges that exhibit excellent wettability and thus enhanced blood absorption capacity without the utilization of wetting agents or blowing agents and with the minimum utilization of crosslinking agents.

### BRIEF SUMMARY OF THE INVENTION

The foregoing paragraphs have been provided by way of general introduction and are not intended to limit the scope of the following embodiment or claims. The described embodiments, together with further advantages, will be best understood by reference to the following detailed description taken in conjunction with the accompanying drawings.

The current disclosure provides a method for manufacturing cross-linked gelatin and gelatin/chitosan foam by controlling the viscosity and the temperature of the solution mixture without the necessity of adding wetting agents, surfactants, or blowing agents or adding significant amounts of these ingredients.

The solution mixture for producing a foam has been formulated to take advantage of a change in viscosity. By precise control over the temperature and the corresponding viscosity the inventors were able to trap air bubbles forming stable foam upon vigorous stirring at critical temperature and viscosity without the need to add surfactants.

The prepared dry foam showed rapid and spontaneous blood/fluid absorption, rapid expansion, filling the wound site, and produced a rapid clotting response.

The disclosed composition can be readily used without prior hydration or wetting, unlike conventional agents like GELFOAM®, GELITA-SPON®, CUTANPLAST®, and show superior fluid absorption up to 60× of the weight of the composition, which is higher than any reported sponge.

Another aspect of this technology is a manufacturing method for hemostatic sponges comprising gelatin and hemostatic sponges comprising gelatin/chitosan, preferably without introduction of extraneous chemicals such as surfactants, wetting agents, hardening agents, or blowing agents.

Other aspects of this technology encompass hemostatic sponges having superior wettability compared to existing hemostatic sponges made with either cross-linked gelatin or crosslinked gelatin in combination with chitosan.

The inventors found that the utilization of such manufacturing method with the exclusion of high concentrations of crosslinkers together with the absence of any hardening agent, surfactants or blowing agents provided the developed hemostatic sponges with superior wettability and enhanced hemostatic activity.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings below.

FIGS. 1A-1H show the superior wettability of the developed gelatin (FIGS. 1C and 1G) and gelatin/chitosan (FIGS. 1D and 1H) sponges as compared to the commercial control sponges (COLTENE® and GELITA-SPON®; FIGS. 1A-qB and FIGS. 1E and 1F). The developed gelatin and gelatin/chitosan sponges allowed instant absorption of the applied liquid (20 uL) at 10 and 20 seconds, hence superior wettability, whereas both the controls showed no fluid absorption and hence very low wettability as apparent from the water droplets remaining on their top surfaces.

FIG. 2. Swelling abilities as demonstrated via the absorption percentage of the sponges over time. The gelatin and gelatin/chitosan sponges of the current invention exhibited enhanced absorption abilities reaching to 60× the weight of the sponge in case of the gelatin/chitosan sponge and around 45× the weight of the sponge in case of the gelatin sponges. Whereas the commercial control sponges (without pre-wetting step) of cross-linked gelatin (Control 1 and Control 2) exhibited much lower absorption abilities.

FIG. 3. Wettability index as demonstrated by full hydration of the sponges of the sponges according to the invention. From left to right the gelatin/chitosan sponge according to the invention (Well 1) was fully hydrated at 8 sec whereas the commercial sponges GELITA-SPON® (Well 2) and CUTANPLAST® (Well 3) demonstrated no hydration for minutes.

FIG. 4. Mechanical properties of G/C sponges (top line). Break stress for G/C was found to be 0.057 N/mm²; which is higher than already marketed products of gelatin hemostat such as GELITA-SPON (bottom line), with a break stress of 0.036 N/mm². Determination of break stress demonstrates the strength of the sponge to withstand any rupture during its application.

### DETAILED DESCRIPTION OF THE INVENTION

The invention provides a method for manufacturing crosslinked gelatin and gelatin/chitosan foam without the necessity of adding wetting agents, surfactants, carbohydrates such as starches, hardening agents, foaming agents, or blowing agents. The inventors attained this by carefully controlling the viscosity and the temperature of the solution mixture used to manufacture the sponge and by taking advantage a change in viscosity of the prepared solution upon decreasing the temperature of the solution mixture. Precise control of the temperature and the corresponding viscosity trapped air bubbles in the mixture upon vigorous stirring produced stable foam within a critical temperature range and within a critical viscosity range thus avoiding the need to add surfactants or other agents to produce stable foam.

Once dried the foam produced a material suitable for inducing hemostasis, with superior wettability, and excellent commercial potential. This material showed rapid and spontaneous blood/fluid absorption, rapid expansion, and was useful for filling a wound site and for inducing a rapid clotting response.

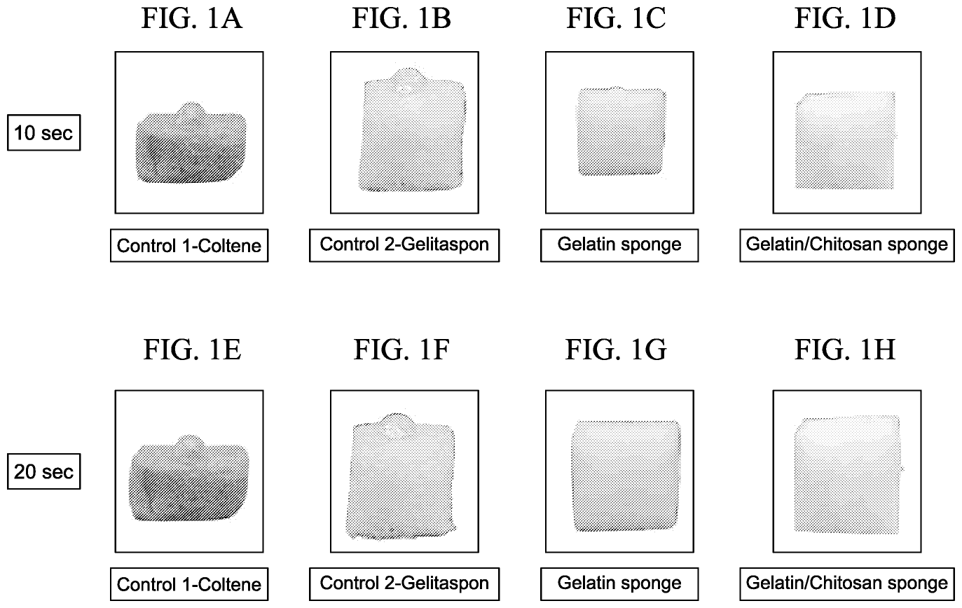

The inventors investigated whether the sponges manufactured via the disclosed manufacturing technique and made of gelatin or gelatin/chitosan would exhibit excellent wettability compared to the commercial gelatin sponges in the market (GELITA-SPON®, CUTANPLAST®, COLTENE®) and whether the inclusion of chitosan would enhance the wettability and the hemostatic activity. It was found that both the developed sponges via the introduced manufacturing technique exhibited superior wettability as compared to the commercial gelatin sponges and improved blood absorption capability. This superior wettability is shown by the controlled comparisons shown in FIGS. 1A-1H. The developed gelatin (FIGS. 1C and 1G) and gelatin/chitosan (FIGS. 1D and 1H) sponges were compared to the commercial control sponges (COLTENE® and GELITA-SPON®; FIGS. 1A-1B and FIGS. 1E-1F). The gelatin and gelatin/chitosan sponges according to the invention allowed instant absorption of the applied liquid (20 uL) at 10 and 20 seconds and exhibited superior wettability, whereas both controls showed no fluid absorption and hence very low wettability. The sponges disclosed herein can be readily used without prior hydration or wetting in contrast to conventional sponges such as COLTENER, GELITA-SPON®, and CUTANPLAST®).

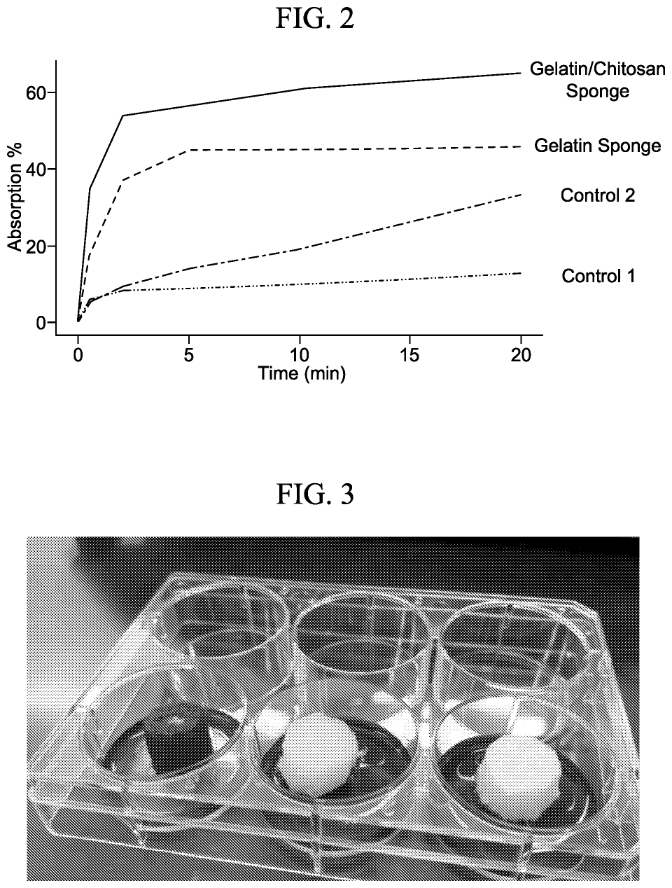

It was found that the inclusion of chitosan allowed an enhancement in wettability and swelling capabilities (blood absorption ability) as compared to the developed gelatin only sponges (FIG. 2). FIG. 2 shows the swelling abilities as demonstrated via the absorption percentage of the sponges. The gelatin and gelatin/chitosan sponges of the current invention exhibited enhanced absorption abilities reaching to 60× the weight of the sponge in case of the gelatin/chitosan sponge and around 45× the weight of the sponge in case of the gelatin sponges. In contrast, the commercial control sponges without a pre-wetting step (Control 1 and Control 2) exhibited much lower absorption abilities.

While not being bound to a particular theory or explanation the inventors attributed this enhanced activity to the very low concentration of cross-linking agents in both the gelatin and gelatin/chitosan sponges, where the porous structure is mainly stabilized via the manufacturing technique rather than by use of high concentrations of crosslinking or blowing agents. Furthermore, the positive formal charge of both chitosan and gelatin can interact with negatively charged red blood cells, thus, inducing platelet aggregation and ultimately stopping bleeding. The inventors also consider that gelatin and chitosan both exhibit other excellent properties including biocompatibility, biodegradability, antimicrobial properties, and non-antigenicity and could confer these on a hemostatic sponge.

One aspect of the disclosure pertains to a method for producing a gelatin or gelatin/chitosan sponge having superior wettability, absorbability or adsorbing ability, and hemostatic properties.

These properties include wettability that is at least >0, 5, 10, 20, 30, 40, 50, 60, 70, 80, 90 or 100% more than a control sponge made from the same ingredients, but containing additional surfactants, foaming agents, wetting agents (such as surfactants), blowing agents, or hardening agents or high concentrations of crosslinking agents not used to produce the sponge disclosed herein.

These properties include an ability of a sponge according to the invention to adsorb or absorb an amount of blood or other fluid that is at least 30, 40, 50, 60, or >60 times the weight of the sponge, for example, over a period of 1, 2, 3, 4, 5, 6, 7, 8, 9, 10, 15 or 20 mins compared to commercial sponges, compared top sponges containing gelatin or gelatin and chitosan, which also contain other ingredients, or compared to sponges with the same or similar ingredients made by other process steps.

One embodiment of this method involves preparing a first aqueous solution of gelatin or gelatin and chitosan by suspending, mixing, or melting it into an aqueous solution at a suitable temperature such as about 30, 35, 40, 45, 50, 55, 60, 61, 62, 63, 64, 65, 66, 67, 68, 69, 70 or >70° C.

The resulting aqueous gelatin or gelatin and chitosan solution is then stabilized in a foam structure via minimum amount of crosslinking and optimal control over the viscosity and temperature.

In another embodiment of this method, the resulting aqueous gelatin solution or gelatin and chitosan solution is acidified by adding about 0.1, 0.2, 0.5, 1.0, 1.5 or 2.0% v/v of acetic acid (glacial acetic acid).

In altemative embodiments, a different organic or inorganic acid such as hydrochloric, ascorbic acid, lactic acid, and citric acid may be used in similar concentrations. Subsequently, the pH of the solution is adjusted to range between pH 3.5. 4.0, 4.5 to pH 5.

In some embodiments, the dried foams have pores ranging in average size from 100, 200, 300, 400, 500, 600, 700, 800, 900, or 1,000 microns. Porosity may be seen in the dried foams of FIG. 3.

An aqueous gelatin solution or acidified gelatin solution contains gelatin in the amount ranging from 0.5, 1, 1.5, 2, 2.5, 3, 3.5, 4, 4.5 and 5 wt. %.

For foams containing gelatin and chitosan, an amount of chitosan is added to the acidified aqueous gelatin solution. Preferably, chitosan is added in an amount ranging from about 0.1, 0.2, 0.5, 1.0, 1.5 or 2.0 wt. % w/v chitosan based on the volume of the acidified gelatin solution.

A w/w ratio of gelatin to chitosan in the resulting solution may range from 20:1, 15:1, 10:1, 5:1 or 4:1.

In an alternative embodiment, the ratio of gelatin to chitosan may range from 1:1, 1:2, 1:3 or 1:4.

The amount of, and ratio of, gelatin and chitosan is selected to provide a viscous solution suitable for producing a stable foam.

The viscosity of the gelatin and gelatin/chitosan solutions varies substantially with temperature with increases in viscosity correlating with lowering temperatures. The foams were fabricated at different temperatures ranging from 15, 18, 20, 25 or 30° C. at different viscosity ranges of 20, 50, 100, 200 and 400 mPa-s.

Crosslinking is induced by addition of, or exposure to, a crosslinking agent, such as an aldehyde like formaldehyde or glutaraldehyde, sufficient to induce crosslinking, for example, an amount of glutaraldehyde (50%) or another aldehyde ranging from about 0.001, 0.002, 0.005, 0.01, 0.02, 0.03, 0.04, 0.05, 0.06, 0.07, 0.08, 0.09, 0.1, or >0.1% (v/v).

In the case of aldehydes, the pH may be held from about 6, 7, 8, 9, 10, to 11, preferably from 7 to 10.

When crosslinking with glutaraldehyde, the crosslinks are formed via Schiff bases which may be stabilized by subsequent reduction, e.g., by treatment with sodium borohydride.

The components of the crosslinked solution of gelatin and gelatin/chitosan are vigorously agitated while the temperature is lowered to 10, 15, to 20° C., to produce a viscous gel and subsequently a foam.

In some embodiments, the rate of temperature decrease is about <0.1, 0.1, 0.2, 0.3, 0.4, 0.5, 0.6, 0.7, 0.8, 0.9, 1, or >1° C. per minute.

In some embodiments, the agitation is continued for about 5, 10, 15, 20, 30, 40, 50 to 60 minutes, preferably about 15 minutes.

After crosslinking and agitation into a foam, the foamy mixture is poured into molds and frozen at a suitable temperature, for example at a temperature from no more than 0, −5, −10, −15, −20 to −80° C. A foamy mixture may also be expressed, sprayed, printed or otherwise deposited in a particular form or on a particular flat or non-flat surface without the use of molds prior to freezing or drying.

After freezing, a frozen molded composition is lyophilized or freeze-dried to produce a dry foam. Compositionally related products to the sponge of the invention may be produced by alternative processing of the foam or dry foam, for example, the dried foam may be pulverized and used as a hemostatic powder.

The process disclosed above for producing a foam is preferably performed without the inclusion or introduction of a wetting agent, surfactant, or blowing agent and solely by controlling the viscosity and temperature of the mixture of cross-linked gelatin and chitosan.

Use of Sponge. The sponges or foams as disclosed herein may be used to pack wounds, absorb blood and stop bleeding. They can be removed after stopping bleeding or left in place to be bio-absorbed over a period of weeks to months. In addition, medicaments, such as antibiotics, growth factors and thrombus enhancing agents, may be incorporated into the cross-linked gelatin and the gelatin/chitosan to enhance the properties of the composition when used therapeutically.

Under use, a hemostatic sponge is placed onto or into a wound, whereupon it absorbs blood or other fluids, can expand to pack and compress the wound, and initiate a rapid clotting response. Unlike conventional cross-linked gelatin sponges, the gelatin and gelatin/chitin-containing sponge disclosed herein may be used without pre-wetting.

Alternatively, if desired or required by a particular surgical procedure, the sponge may be pre-hydrated, wetted or washed in a sterile fluid like normal saline or fluid containing additional active ingredients before placement in a wound.

A sponge can be compressed by hand and inserted or tamped into a wound.

In one embodiment, a sponge is used to treat a puncture site such as a puncture wound resulting from catheter insertion or a biopsy needle.

In another mode of administration, a sponge is ejected from a syringe into a puncture site such as a punctured artery or vein. Such a puncture may occur during coronary angioplasty, angiography, atherectomy, stenting of arteries, and many other procedures which involve accessing the vasculature through a catheter placed in the femoral artery or other blood vessel. Once the procedure is completed and the catheter or other instrumentation is removed, bleeding from the punctured artery must be controlled. These and other wounds and modes of administration are described by and incorporated by reference to Cragg, et al., U.S. Pat. No. 6,071,301, Device and Method for Facilitating Hemostasis of a Biopsy Tract, issued Jun. 6, 2000. Cragg, et al., U.S. Pat. No. 6,086,607, Device and Method for Facilitating Hemostasis of a Biopsy Tract, issued Jul. 11, 2000; Cragg, et al., U.S. Pat. No. 6,162,192, System and Method for Facilitating Hemostasis of Blood Vessel Punctures with Absorbable Sponge, issued Dec. 19, 2000.

Alternative uses. The sponges and materials made by the methods disclosed have a variety of uses in addition to their uses in medical procedures as hemostatic sponges. Other uses include absorbent articles for intake, distribution, and retention of human body fluids. Examples include feminine care pads, tampons, diapers, incontinence articles, training pants, bed pads, sweat absorbing pads, shoe pads, bandages, helmet liners, wipes and wipers, etc., or, in a suitably thin and flexible form, as a novel tissue or towel.

The sponges and related materials disclosed herein can maintain a three-dimensional structure and maintain stability under stress and when wet. Thus, a wide variety of shaped composites can be envisioned, including tampons, shock-absorbing shoe pads, articles adapted for particular portions of garments or the body, gaskets for ostomy bags, hemostatic sponges and other medical sponges and absorbents for surgical purposes, dental absorbents such as plugs for extracted teeth or saliva absorbents to fit in portions of the mouth, and the like.

Besides serving as absorbent articles, materials of the present invention can serve as components in filters, including filters for absorbing liquid droplets and other entrained materials in the air, including face masks. Filters made with the absorbent fibrous structures disclosed herein can be particularly useful when they comprise activated carbon fibers or granules. Such filtration materials are capable of absorbing pollutants or odors from gases and organic pollutants from liquids, particularly water. The absorbent fibrous structures of the present invention can also be used in additional products such as shock absorbing pads, groundcover materials, erosion barriers, pads for absorbing pet waste, industrial spill and leak absorbents, floating barriers for oil spills and chemical containment, fireproofing materials, insulation, packaging materials, padding, and the like. The absorbent fibrous structures of the present invention can also be combined with other functional materials internally (as by adding material into the absorbent fibrous structure) or externally (as by joining with additional layers) such as odor absorbents, activated carbon materials, fire retardants, superabsorbent particles, nonwoven materials, plastic films or apertured films, extruded webs, closed cell foams, tissue webs, electronic devices such as alarms indicating wetness or leakage, opacifiers, fillers, aerogels, sizing agents, antimicrobial agents, adhesive strips and tapes, and the like.

The term “sponge” includes sponges, pledgets (a small wad of absorbent cotton or other soft material used to stop up a wound or other opening in the body), tampons, and flat absorbent pads, especially those used to medicate, protect to adsorb or absorb drainage from a wound or sore. A “hemostatic sponge” is used to control blood loss or promote coagulation or clotting. A “surgical sponge” may be used before, during, or after a surgical procedure,

The term “gelatin” or “gelatine” is well defined in the art and encompasses its usual meaning. It typically refers to a translucent, colorless, flavorless food ingredient, commonly derived from collagen taken from animal body parts. It is brittle when dry and gummy when moist. It may also be referred to as hydrolyzed collagen, collagen hydrolysate, gelatine hydrolysate, hydrolyzed gelatine, and collagen peptides after it has undergone hydrolysis. Gelatin is classified as a hydrogel. Gelatin absorbs 5-10 times its weight in water to form a gel. Gelatin is a collection of peptides and proteins produced by partial hydrolysis of collagen extracted from the skin, bones, and connective tissues of animals such as domesticated cattle, chicken, pigs, and fish. Gelatins have diverse melting points and gelation temperatures, depending on the source. For example, gelatin derived from fish has a lower melting and gelation point than gelatin derived from beef or pork. Gelatin of human origin can be obtained through two major steps, extracellular matrix (ECM) extraction from human adipose tissue and gelatin isolation from the ECM or other collagen-containing tissues or fluids. In some embodiments, gelatin may be obtained by processing collagen produced in vitro, for example, by culture of fibroblasts or other cells which produce collagen. The tyrosine residues in human gelatin can be converted into DOPA by enzymatic reaction with tyrosinase. Upon the addition of Fe³⁺ ions, the DOPA-modified gelatin formed a sticky hydrogel within seconds through complexation between the DOPA molecules and Fe³⁺ ions.

Collagen, from which gelatin can be made, is the main structural protein in the extracellular matrix found in the body's various connective tissues. As the main component of connective tissue, it is the most abundant protein in mammals, making up from 25% to 35% of the whole-body protein content. Collagen consists of amino acids bound together to form a triple helix of elongated fibrils known as a collagen helix. It is mostly found in connective tissue such as cartilage, bones, tendons, ligaments, and skin. Over 90% of the collagen in the human body is type I collagen. However, as of 2011, 28 types of human collagen have been identified, described, and divided into several groups according to the structure they form. All of the types contain at least one triple helix. The number of types shows collagen's diverse functionality. Collagen types include Fibrillar (Type I, II, III, V, XI) collagen, Non-fibrillar collagens including FACIT (Fibril Associated Collagens with Interrupted Triple Helices) (Type IX, XII, XIV, XIX, XXI); Short chain (Type VIII, X); Basement membrane (Type IV); Multiplexin (Multiple Triple Helix domains with Interruptions) (Type XV, XVIII); MACIT (Membrane Associated Collagens with Interrupted Triple Helices) (Type XIII, XVII); Microfibril forming (Type VI); and Anchoring fibrils (Type VII). The five most common types of collagen are: Type I found in skin, tendon, vasculature, organs, and bone (main component of the organic part of bone); Type II: cartilage (main collagenous component of cartilage); Type III: reticulate (main component of reticular fibers), commonly found alongside type I; Type IV: forms basal lamina, the epithelium-secreted layer of the basement membrane; and Type V collagen found on cell surfaces, hair, and placenta.

Gelatin can be classified into type A and type B, based on acid and alkaline pretreatment, respectively. Type A gelatin is created by treating collagen with acid. It is derived from pig skin, fish skin/scales, beef hides or beef bones and is typically used in food-grade products. It has a lower viscosity but higher bloom than Type B. Type B gelatin is manufactured using a high-pH treatment and is most often derived from pig skin, fish skin/scales, beef hides or beef bones. Type B alkaline gelatin is generally for industrial uses. It has a higher viscosity and lower bloom strength. A suitable form of gelatin may be selected for production of the sponges disclosed herein.

Bloom value. The gelatin bloom value or number indicates the firmness or softness of a specific type of gelatin and is mainly related to its viscosity. Gelatin viscosity determines the texture and creaminess of the final product. The bloom strength of gelatin can range from 30 to 325 with 30 being the softest and 325 being the stiffest. Bloom strength ranges tend to fall into three categories.

Low Bloom Gelatin. Low bloom gelatin falls between the range of 50-125. Lower bloom gelatin comes from the early stages of the gelatin-making process. Since little time goes into making it, low bloom gelatin is very soft.

Medium Bloom Gelatin. Medium bloom gelatins range between 175-225 in bloom strength. Gelatins in this range are often used for food. For example, gelatin with a bloom of 225 can be found in frosting, whipped cream stabilizers, marshmallows and canned ham.

High Bloom Gelatin. High bloom gelatin ranges from 225-325 and is often made from cow or pig collagen. It can be formulated to create a firm transparent gel. High bloom gelatin is most often used in gelatin desserts, jelly fillings, cream fillings, jellied meat products, soft gelatin capsules and ballistic gelatin.

High Bloom bovine gelatin (>240 g Bloom) is preferably utilized for the sponges. Low endotoxin gelatine provides excellent compatibility with the body. Gelatine is neutral in taste and odor and is practically non-allergenic.

Differences between gelatin and collagen. Gelatin and collagen are both protein substances which contain numerous glycine, proline, hydroxyproline residue groups. Collagen is complex in structure and consists of three helical chains. Gelatin is the broken down form of collagen consisting of smaller peptides of amino acids. Gelatin is formed synthetically from collagen through a series of reactions that involve alkalines or acids and boiling treatments. Collagen is the most abundant connective tissue fiber that is formed and found in the human body. The gelatin is useful in the food industry and as a thickening agent. The collagen is used in medical or surgical restorations. Gelatins produced from the various collagens described herein may be used as components in the sponge, pledgets, and other products disclosed herein.

The term “chitosan” refers to a linear polysaccharide composed of randomly distributed β-(1→4)-linked D-glucosamine (deacetylated unit) and N-acetyl-D-glucosamine (acetylated unit). It is made by treating the chitin shells of shrimp and other crustaceans with an alkaline substance, such as sodium hydroxide. Chitosan is dissolved in dilute organic acid solutions but is insoluble in high concentrations of hydrogen ions at pH 6.5 and is precipitated as a gel-like compound. Chitosan is positively charged by amine groups, making it suitable for binding to negatively charged molecules. However, it has disadvantages such as a low mechanical strength and low-temperature response rate.

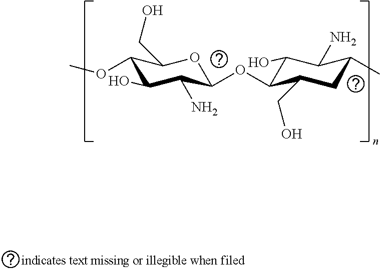

A preferred source of our chitosan is chitosan from shrimp shell was from a certified company that works according to Hazards Analysis Critical Control Point Program (HACCP) to assure product safety and quality. Our utilized chitosan is ISO22000 certified which means written procedures are provided for all manufacturing steps and for each specification parameter. Various analyses are performed through whole production and Certificate of Analysis is issued for every final batch produced. The properties of the utilized chitosan are (Mw, 240 kDa and DDA of 84%, cg110, TM 3728). The utilized chitosan exhibits positive charge thus attracts negatively charged red blood cells, making an efficient hemostatic agent. For alternative chitosans, these values may range downward or upward by at least, or no more than, 1, 2, 5, 10 or 20%.

Crosslinking may be induced by exposure to chemical agents, such as formaldehyde, glutaraldehyde, or other aldehydes by peroxides, by sulfanic acids, by glyoxal, or by other chemical crosslinkers known in the art, such as those described by, and incorporated by reference to, Double-Do Protein Cross-Linkers Handbook & Selection Guide, available at hypertext transfer protocol://wolfson.huji.ac.il/purification/PDF/ProteinInteractions/GBIOSC_ ProtCrossLinkersHandbook.pdf (last accessed Sep. 20, 2021). Crosslinking may be induced or may occur between gelatin molecules and other gelatin molecules or between gelatin molecules and chitosan molecules or other known chemical crosslinkers.

Wettability index. As used herein, a “wettability index” refers to the time it takes for a sample of known dimension to fully hydrate. A preferred embodiment of the invention which are illustrated in the Examples has a wettability index of less than or equal to 1 minute in distilled water at room temperature (FIG. 3).

FIG. 3 shows the wettability index as demonstrated via the full hydration of the sponges. From left to right the developed gelatin/chitosan sponge was fill hydrated at 8 sec whereas the commercial sponges GELITA-SPON® and CUTANPLAST® demonstrated no hydration for minutes.

Additionally, contact angel testing was performed as a direct indicator of the wettability of the developed sponges. A drop of 20 μL water was tested for solid surface wettability. Different angles represent the surface hydrophobicity where θ≥90° represent hydrophobic surfaces, and θ<90° represent hydrophilic surfaces (Table 1).

Table 1: Measure of the contact angels at two time intervals (10 sec and 20 sec) of the developed gelatin and gelatin/chitosan sponges as compared to the controls (GELITA-SPON® and COLTENE®).

| Contact | Gelatin/ | | | |
|---|---|---|---|---|
| Angle | Chitosan | Gelatin | Control 1 | Control 2 |
| (⊖) | Sponge | Sponge | (GelitaSpon ®) | (Coltene ®) |
| 10 sec | 12° | 79.2° | 104° | 131.2° |
| 20 sec | 0° | 55.2° | 101° | 130° |

Gelatin sponges were produced using the methods disclosed herein. They demonstrated a contact angel of 79.2° and 55.2° at 10 sec and 20 sec respectively thus indicating very high hydrophilicity as compared to the controls which demonstrated contact angels much more than 90° thus indicating hydrophobic nature. Interestingly the inclusion of chitosan allowed very high increase in the wettability where the contact angle was 12° at 10 sec and reached to 0° at 20 sec (FIG. 1A-1H)

FIG. 1 demonstrates the superior wettability of the developed gelatin and gelatin/chitosan sponges as compared to the commercial control sponges (GELITA-SPON® and COLTENE®). The developed gelatin and gelatin/chitosan sponges allowed instant absorption of the applied liquid (20 ul) at 10 and 20 seconds, hence superior wettability, whereas both the control sponges showed no fluid absorption as apparent from the water droplets on the tops of the sponges, and hence very low wettability.

Hemostatic activity. Hemostatic activity may be measured by known assays such as those described by, and incorporated by reference to, Handbook of diagnostic Hemostatis and Thrombosis Tests; University of Washington (2005) available at hypertext transfer protocol://depts.washington.edu/labweb/PatientCare/Clinical/Guides/hemostasis.pdf (last accessed Sep. 21, 2021). The hemostatic activity of the sponges was evaluated utilizing two methods a qualitative method using blood smear aggregation and a quantitative method of platelet count aggregation.

Blood smear aggregation. 100 μL of whole blood was aspirated on glass slide. The sponges were placed on top of the blood and left to stand for 3 min. The slide is then smeared and left to dry. Absolute ethanol is utilized as a fixing media for 5 minutes. Leichmann stain is used to cover the whole slide for 15 minutes then washed with tap water and left to dry. The micrograph images are taken by Olympus microscope at 10× magnification.

Platelet aggregation. Quantification test of platelet is taken place on PRP where 100 μL is aspirated in each well. The chitosan/PVA sponges are excised at 0.5×0.5 cm sterilized and placed in each well for 3 min taking the count of platelets at time 0 min, 1 min and 3 mins. At each time point, 10 μL is aspirated and placed in ammonium oxalate in ratio of 1:19 and left for 5 min. Then 10 μL is loaded in the hemocytometer on both chambers to be counted twice for each sample per time point. The hemocytometer is placed in a humidified chamber and left for 15 minutes to settle the platelets. The platelets are counted and calculated as follow:

$$\frac{plateletcount}{100 µL ( well )} = \frac{count}{5} \times \frac{1}{0.2} \times 20 \times 100 \qquad ( Eq . 3 )$$

Tensile strength. The tensile strength of the hemostatic sponge of the invention can be compared to the tensile strength of control sponges made of crosslinked gelatin without chitosan or made by a similar process but with surfactants, foaming agents, carbohydrates, wetting agents (such as surfactants), blowing agents, or hardening agents not required for production of the gelatin or gelatin/chitosan sponges disclosed herein.

A tensile test measures the level of strength that a material possesses. A material testing laboratory usually conducts the test using a universal testing machine (UTM), which holds a specimen material in place and applies the tension stress needed to check the breaking point. Advantageously, the hemostatic sponge of the invention may have an increase in tensile strength compared to a control sponge of at least 1, 2, 5, 10, 15, 20, 25 or 30%. A control sponge typically contains the same ingredients as a sponge as disclosed herein except that it omits chitosan. A control sponge may substitute an equivalent weight of gelatin for the omitted chitosan. A control sponge may be made of gelatin but with the addition of foaming agents, carbohydrates, wetting agents (such as surfactants), blowing agents, or hardening agents. Comparisons of the gelatin and gelatin/chitosan-containing sponges disclosed herein may also be made to sponges comprising other ingredients, such as conventional sponges containing crosslinked gelatin as well as other ingredients.

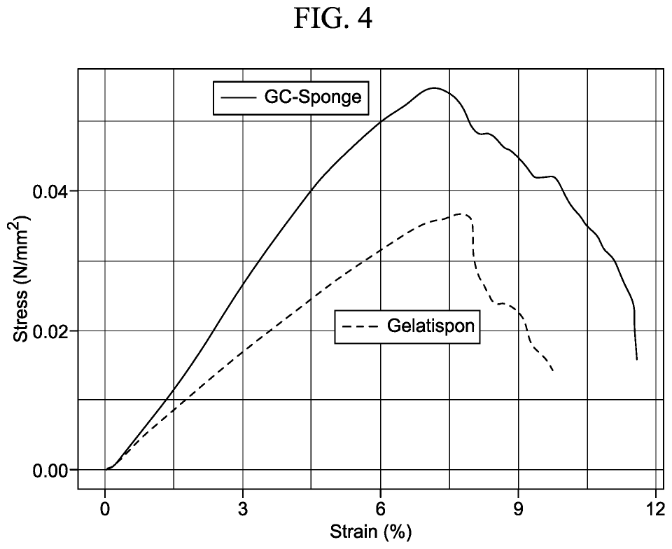

Mechanical properties of sponges were tested using the UTM (Universal Testing Machine) instrument (Instron 3342) with 100 Newton (N) of load cell. sponges were cut into rectangular shaped (10 cm×0.5 cm) strips and their ends clamped with steel grip jaws of UTM instrument for the measurement. FIG. 4 depicts mechanical properties of G/C sponges. Break stress for G/C was found to be 0.057 N/mm²; which is higher than already marketed products of gelatin hemostat such as GELITA-SPON®, with a break stress of 0.036 N/mm². Determination of break stress demonstrates the strength of the sponge to withstand any rupture during its application.

Surfactants include cationic, non-ionic, zwitterionic, and anionic surfactants. Examples include sodium lauryl sulfate (SDS), cetyl trimethylammonium bromide (CTAB), Tritons such as Triton X-100 or Triton X-114, CHAPS, DOC, NP-40, octyl thioglucoside, octyl glucoside and dodecyl maltoside. Surfactants are compounds that lower the surface tension (or interfacial tension) between two liquids, between a gas and a liquid, or between a liquid and a solid. Surfactants may act as detergents, wetting agents, emulsifiers, foaming agents, or dispersants. In preferred embodiments. surfactants, or additional surfactants, are not used to produce the gelatin/chitosan sponge but may be used to produce a gelatin only sponge.

Foaming agents include but are not limited to surfactants such as sodium laureth sulfate, cocamides such as nonionic cocamide DEA and cocamidoproylamine oxide, and zwitterionic cocamidopropyl betaine and cacamidoproply hydroxysultaine and gases generated by chemical reactions such as by baking powder, azodicarbonamide, titanium hydride, and isocyanates (when reacted with water). In preferred embodiments, foaming, or additional foaming agents, are not used to produce the sponge or other related products disclosed herein.

Wetting agents include anionic, cationic and amphoteric agents which ionize when mixed with water and non-ionic wetting agents which do not ionize. A surfactant may serve as a wetting agent. Wetting agents are known in the art and are commercially available, for example, as described by, and incorporated by reference to, Dow Surfactants Reference Chart, hypertext transfer protocol secure ://www.dow.com/content/dam/dcc/documents/en-us/catalog-selguide/119/119-01491-01-dow-surfactants-selection-guide.pdf?iframe=true (last accessed Sep. 21, 2021). Other biocompatible wetting agents are commercially available and include, for example, sodium lauryl sulfate, Pluronic™ F-68, Pluronic™ F-38, Pluronic™0 P-105, Pluronic™-10R5, Tween™ 20, Tween™ 60, Tween™ 85, Brij™ 35, Brij™ 78, Myrj™ 52, PEG™ 600, glycerin and the like. In preferred embodiments, wetting agents, or additional wetting agents, are not used to produce the sponge or other related products disclosed herein.

Blowing agents include physical blowing agents such as the gases carbon dioxide, pentane, and chlorofluorcarbons and chemical blowing agents such as isocyanate and water, azodicarbonamide, and sodium bicarbonate which produce gases by reaction with water or other materials. In preferred embodiments, blowing agents, or additional blowing agents, are not used to produce the sponge or other related products disclosed herein.

Hardening agents showing a high hardening reaction for gelatin and having an excellent water solubility include N-carbamoylpyridinium salts as described in Japanese Patent Application (OPI) Nos. 51,945/'74 and 59,625/'76 corresponding respectively to U.K. Pat. No. 1,383,630 and to U.S. Pat. No. 4,063,952; and 2-sulfonyloxypyridinium salts as described in Japanese Patent Application (OPI) No. 110,762/'81. These hardening agents having features such that a water solubility is high, the hardening action for gelatin is fast, and the occurrence of post-hardening is less. In preferred embodiments, hardening agents, or additional hardening agents, are not used to produce the sponge or other related products disclosed herein.

Antioxidants. In some embodiments of the sponge or related materials one or more antioxidants may be incorporated. These include, but are not limited to, a tocopherol, such as alpha-, beta-, gamma- or delta-tocopherol, mixtures thereof, or other antioxidants such as tocotrienols, resveratrol or other stilbenoids such as pterostilbene, retinoids and carotenes including Vitamin A, beta carotene, and alpha-carotene, astaxanthin, canthaxanthin, lutein, lycopene, and zeaxanthin, natural phenols including flavonoids, silymarin, xanthones, eugenol, phenolic acids, Vitamin C, lipoic acid, acetylcysteine, uric acid, carotenes, glutathione, catalase, superoxide dismutase, manganese, selenium, may be included in or on a sponge or related material disclosed herein. Antioxidants may be incorporated to facilitate or control the rate of wound healing or to prevent scarring. In other embodiments, no antioxidant is included.

Biologically active peptides include antibiotic peptides. bacterial peptides, brain peptides, cancer and anticancer peptides, cardiovascular peptides, clotting or coagulating peptides, anticoagulant or antithrombotic peptides or proteins, and regenerative peptides.

Antibiotics include bacteriostatic and bactericidal agents. Classes of antibiotics include aminoglycosides, ansamycins, carbacephems, carbapenems, cephalosporins (first, second, third, fourth and fifth generations), glycopeptides, lincosamides, lipopeptides, macrolides, monobactams, nitrofurans, oxazolidinones, penicillins, penicillin combinations, polypeptides including antimicrobial peptides, quionoles/fluroquinolones, sulfonamides, tetracyclines, antimycobacterial drugs, and other drugs, such as arsphenamine, chloramphenicol, fosfomycine, fusidic acid, metronidazole, mupirocin, platensimycin, quinupristin/dalfopristin, tigecycline, tinidazole, and trimethoprim. Specific antibiotics within these classes are known and are incorporated by reference to hypertext transfer protocol secure://en.wikipedia.org/wiki/List_of_antibiotics (last accessed Sep. 21, 2021). One or more antibiotics may be incorporated into the sponges and related products disclosed herein. In other embodiments one or more antibiotics may be excluded from a sponge or related product.

Drugs affecting hemostasis includes drugs which facilitate clotting. These include antifibrinolytic drugs such as aprotinin, tranexamic acid (TXA), epsilon-aminocaproic acid, and amino methylbenzoic acid.

In some alternative embodiments drugs which inhibit clotting or thrombosis may be incorporated, such as Class IC antiarrhytmics, such as flecainide, cyclooxygenase inhibitors, such as aspirin, ADP receptor pathway inhibitors such as clopidogrel, anticoagulants such as warfarin, and selective thrombin inhibitors such as dabigatran.

### EMBODIMENTS

Embodiments of the disclosure include but are not limited to the following.

A method for manufacturing a hemostatic sponge comprising, consisting essentially of, or consisting of, contacting acidified gelatin and chitosan to form an aqueous solution in the presence of a crosslinking agent to form a mixture, forming a foam from the mixture, and freezing the foam, and lyophilizing the frozen foam to form the hemostatic sponge. In some embodiments, the solution lacks chitosan and is made only of aqueous gelatin with a crosslinker. In other embodiments the foam may be desiccated without freezing, converted to particles or a powder, or formed into a sheet and thus form a compositionally related product to the hemostatic sponges disclosed herein. Preferably, the method disclosed above is performed without incorporating a surfactant, wetting agent, hardening agent, or blowing agent into the mixture or into the final hemostatic sponge or other product.

In the method disclosed above, a w/w ratio of gelatin to chitosan in the aqueous solution ranges from about 20:1. 15:1. 10:1, 5:1 or 4:1, or other ratio suitable for producing a viscous foamable solution.

In the methods disclosed herein, the gelatin may be that described by CAS Number 9000-70-8 and/or the chitosan that described by CAS Number 9012-76-4.

In some embodiments of the methods disclosed herein, the acidified gelatin is acidified by addition of 0.1, 0.2, 0.3, 0.4, 0.5, 0.6, 0.7 to 0.8% v/v acetic acid, ascorbic acid, lactic acid, citric acid or hydrochloric acid to an aqueous gelatin solution.

In some embodiments of the methods disclosed herein the crosslinking agent is a dialdehyde in an amount ranging from 0.01, 0.02, 0.03, 0.04, 0.05, 0.06, 0.07, 0.08, 0.09 to 0.1% v/v.

Crosslinking of gelatinous and polysaccharide materials with glutaraldehyde (GTA) involves the reaction of amino/hydroxy groups, respectively, with the aldehyde groups of GTA. Biomedical wide utilization of GTA crosslinked substrates such as bioprosthetic indicates the clinical acceptance for practice despite of its concentration dependent cytotoxicity. It has been reported that glutaraldehyde concentration up to 8% is non-toxic, which is 80 folds higher than the highest concentration used in different embodiments fabrication.

In some embodiments of the methods disclosed herein the forming the foam comprises, consists essentially of, or consists of vigorous mixing of the mixture for 5, 10, 15, 20, 25 to 30 mins while reducing the temperature of the mixture to 15, 16, 17, 18, 19-20° C. Tin some embodiments, mixing into a foam is performed using a sonicator, blender, or other mechanical mixer.

The methods disclosed herein may optionally incorporate other agent into a hemostatic sponge or compositionally related product, such as at least one antibiotic, antioxidant, biologically active peptide, or drug.

Another aspect of this technology is directed to a hemostatic sponge or compositionally related product made by the methods disclosed herein.

Another aspect of this technology is directed to a hemostatic sponge or compositionally related product made by crosslinked gelatin only and made by the methods disclosed herein.

Another aspect of the disclosure is directed to a hemostatic sponge comprising, consisting essentially of, or consisting of, crosslinked gelatin and chitosan having a wettability greater than that of the control sponge not made with the chitosan, and/or having hemostatic properties greater than the control hemostatic sponge made without chitosan or made with a replacement of the amount of chitosan with gelatin. In other embodiments the tensile strength, wettability or hemostatic properties may be compared with those of conventional hemostatic sponges.

The hemostatic sponge and compositionally related products may be produced from different types of gelatin and chitosan. In some embodiments, the gelatin will conform that the described by CAS Number 9000-70-8 and/or the chitosan to that described by CAS Number 9012-76-4. Other types of gelatin, such as from bovine or other known sources may be used. In some instances, gelatin having the same amino acid sequence or amino acid content may be used. Human gelatin obtained from cell culture or other acceptable sources may be used in some embodiments. Various types of gelatin may be selected such as type A or type B gelatins and various types of chitosan such as alpha- or beta-chitosan, may be used to produce the sponges and compositionally related products disclosed herein.

In some embodiments the hemostatic sponge is produced from foam made using a sonicator, blender, stirrer, vortex, frother or foamer (such as a milk foamer or frother), fluid gel foamer, agitator, whipper, beater, or other mechanical mixer.

Another aspect of the disclosure is directed to a procedure, such as a medical or dental procedure, or surgery, comprising, consisting essentially of, or consisting of, contacting the hemostatic sponge or compositionally related product as disclosed above with a wound or a site of bleeding on or in the body of a patient in need thereof. Such a patient may be a human, mammal or other animal benefiting from the hemostatic properties of the sponge. Thus the sponges and compositionally related materials may be used in human clinical medicine as well as in veterinary medicine, for example, to treat cattle, horses, camelids, goats, sheep, dogs, cats, falcons, and other mammals and avians. In one embodiment, the hemostatic sponge is used after phlebotomy, blood donation or transfusion, or other injection, such as after vaccination.

In some procedures, the hemostatic sponge is contacted with a puncture in the vascular system; with a site of inter-cavity bleeding or with a deep wound; with a site on an internal organ or tissue or in the gastrointestinal tract; with a site in the head, spinal canal, chest, or abdomen; with an epidermal wound; with a mucosal wound; with a wound or site of bleeding in the nasal cavity; with a wound or site of bleeding in mouth; with a sports wound or injury; with an animal or insect bite; or with a gunshot, blast, shrapnel, or other combat wound. Wounds obtained in other occupations such as law enforcement, construction, sanitation, medicine may also be treated. In some embodiments, the hemostatic sponge or a compositionally related product may be incorporated into a garment or other device worn on the body, including a bullet proof vest, underwear, shirt, trousers, hat, socks or shoes.

The following examples are set forth to illustrate the embodiment/claimed invention and are not to be construed as a limitation thereof.

### EXAMPLE 1

Two main factors were tested in the fabrication process. The initial concentrations of the two main components in addition to the crosslinking concentration. The concentration of the gelatin was tested between 3-5% (w/v) with chitosan 0.2-2% (w/v) and the crosslinker concentration was optimized for the range of 0.01-0.1% (v/v). After selecting the concentration ratios, the foam structure formation was evaluated at different fabrication temperatures at 37° C., 25° C., and 18° C. with the aim of preparing a stable foam prior to forming a dried foam structure by drying the foam with a lyophilizer.

Physicochemical characterizations were assessed using FTIR, TGA, SEM, and degradation study. Biological evaluations also were carried out to evaluate the biocompatibility of the sponges and their capability to induce platelet aggregation and hemostasis of whole blood upon bleeding.

Sponges were prepared as follows.

A stock solution of 3% gelatin was prepared at 60° C. with continuous stirring.

Then acetic acid was added to reach a final concentration of 0.2% v/v.

To prepare 0.2% (w/v) chitosan solution, chitosan powder was added to the above-prescribed solution with constant stirring.

Aided with mechanical stirrer, dialdehyde-based crosslinker was added under vigorous agitation.

Foam was produced by maintaining vigorous stirring while lowering solution temperature to reach a final temperature of 18° C. The solution was stirred for 15 min.

The resulting mixture was poured into molds and left at −20° C. overnight.

Dry foam was obtained after lyophilization for 48 h. To study the effect of solution temperature on the prepared foam, different samples were prepared at different temperatures 37° C. (a), 25° C. (b), and 18° C. (c).

The characteristics of the yielded sponges varied at different temperatures, especially when the solution temperature reached 18° C. (c). Preparation at this temperature resulted in a low density, highly interconnected polygonal uniformly distributed porous structure which showed a superior swelling ratio that reached up to 60× the initial weight of the sponge and/or which could induce or promote rapid platelet aggregation.

On the other hand, preparations at 37° C. and 25° C. resulted in differences in the pore size, distribution, and inner structure. The differences could be interpreted by the fact that the viscosity of the mixture increased with lowering the solution temperature.

Such process enabled the fabrication of stable gel foam of gelatin/chitosan sponge without the need of adding foaming agent, wetting agents (such as surfactants), or hardening agents.

During the fabrication process, different active ingredients may be incorporated such as proteins, active peptide, coagulating agents, antibiotics, and growth factors or other agents and compounds disclosed herein.

### EXAMPLE 2

Two main factors were tested in the fabrication process. The initial concentrations of gelatin in addition to the crosslinking concentration. The concentration of the gelatin was tested between 3-5% (w/v) and the crosslinker concentration was optimized for the range of 0.01-0.1% (v/v). After selecting the concentration ratios, the foam structure formation was evaluated at different fabrication temperatures at 37° C., 25° C., and 18° C. with the aim of preparing a stable foam prior to forming a dried foam structure by drying the foam with a lyophilizer.

Sponges were prepared as follows.

A stock solution of 3% gelatin was prepared at 60° C. with continuous stirring.

Aided with mechanical stirrer, dialdehyde-based crosslinker was added under vigorous agitation.

Foam was produced by maintaining vigorous stirring while lowering solution temperature to reach a final temperature of 18° C. The solution was stirred for 15 min.

The resulting mixture was poured into molds and left at −20° C. overnight.

Dry foam was obtained after lyophilization for 48 h. To study the effect of solution temperature on the prepared foam, different samples were prepared at different temperatures 37° C. (a), 25° C. (b), and 18° C. (c).

The characteristics of the yielded sponges varied at different temperatures, especially when the solution temperature reached 18° C. (c). Preparation at this temperature resulted in a low density, highly interconnected polygonal uniformly distributed porous structure which showed a superior swelling ratio that reached up to 50× the initial weight of the sponge and/or which could induce or promote rapid platelet aggregation.

On the other hand, preparations at 37° C. and 25° C. resulted in differences in the pore size, distribution, and inner structure. The differences could be interpreted by the fact that the viscosity of the mixture increased with lowering the solution temperature.

Such a process enabled the fabrication of stable gel foam of gelatin hardening agents.

During the fabrication process, different active ingredients may be incorporated such as proteins, active peptides, coagulating agents, antibiotics, and growth factors, or other agents and compounds disclosed herein.

The novel sponges and the innovative process of fabrication enable local manufacturing of hemostatic sponges and gauzes in Egypt/MENA/Africa and allow competition with imported hemostatic sponges now on the market. The disclosed sponges can target the surgical, dental and trauma as well as the military sectors in the MENA region. This can be achieved via direct sales to dental clinics, hospitals, military and fire stations. Moreover, sponges such as nasal tampons, bandages, and sponges for topical use, can be sold in pharmacies and used directly by consumers.

Surgical procedures need fast and effective hemostasis and the disclosed sponges can be designed and produced in a range of sizes that allow them to fit to different surgical wounds. The sponges can easily be cut into different sizes. Nasal bleeding is a common problem that is often associated with inconvenience and discomfort. Thus, the introduced sponges can also be advantageously used as nasal tampons “NT-Sponges”. NT-sponge turn are designed to turn into a viscous gel upon contact with the blood which activates the clotting system and leads to moist wound environment that accelerate the re-epithelization of the nasal mucosa. Such tampons are easily removed, thus do not cause any second damage to the wound.

Other features of the sponge. A sponge as disclosed herein will have one or more, preferably all, of the following features. It is non-toxic, does not cause allergic reactions, lacks extraneous chemical compounds not necessary for stopping blood flow, bas a neutral or near neutral pH, absorbs 30-70 times its weight in blood, may be applied dry, may be applied wet or hydrated, may be soaked in a sterile solution containing antibiotics or other active agents, can be reduced in size or custom cut or sized for a particular wound or procedure, has a reduced lead time for use in patients compared to conventional sponges requiring prehydration, reduces the time period of a surgical procedure or intervention, stops hemorrhaging immediately or within no more than 1, 2, 3, 4, 5, 6, 7, 8, 9 or 10 minutes, and when placed in vivo dissolves within 3 to 4 weeks.

Terminology. Terminology used herein is for the purpose of describing particular embodiments only and is not intended to be limiting of the invention.

The headings (such as “Background” and “Summary”) and sub-headings used herein are intended only for general organization of topics within the present invention, and are not intended to limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

While aspects of the present disclosure have been described in conjunction with the specific embodiments thereof that are proposed as examples, alternatives, modifications, and variations to the examples may be made.

Unless expressly stated, the terms used herein are intended to have the plain and ordinary meaning as understood by those of ordinary skill in the art.

The following definitions are intended to aid the reader in understanding the present disclosure, but are not intended to vary or otherwise limit the meaning of such terms unless specifically indicated.

It should be noted that, as used in the specification and the appended embodiment/claims, the singular forms “a,” “an,” and “the” include plural referents unless the context clearly dictates otherwise.

As used herein, the term “and/or” includes any and all combinations of one or more of the associated listed items and may be abbreviated as “/”.

As used herein in the specification and embodiment/claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions.

A numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), or +/−20% of the stated value (or range of values).

Any numerical range recited herein is intended to include all sub-ranges subsumed therein. where a range of values is provided, it is to be understood that each intervening value between an upper and lower limit of the range and any other stated or intervening value in that stated range is encompassed within the disclosure. Where the stated range includes upper and lower limits, ranges excluding either of those limits are also included.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be embodiment/claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be embodiment/claimed using endpoints of the disclosed ranges. For example, if parameter X is exemplified herein to have values in the range of 1-10 it also describes subranges for Parameter X including 1-2, 1-3, 1-4, 1-5, 1-6, 1-7, 1-8, 1-9, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, 9-10 as some examples. A range encompasses its endpoints as well as values inside of an endpoint, for example, the range 0-5 includes 0, >0, 1, 2, 3, 4, <5 and 5.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Materials or products identified by trademark include those that are commercially available on our about the filing date of this application. For example, a trademarked material may refer to a product commercially available 0, 6, 12, 18 or 24 months prior to the present filing date.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Spatially relative terms, such as “under”, “below”, “lower”, “over”, “upper”, “in front of” or “behind” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references and does not constitute an admission as to the accuracy of the content of such references.

1-71. (canceled)

72. A method for manufacturing a hemostatic sponge comprising:
contacting acidified gelatin and chitosan to form an aqueous solution in the presence of a crosslinking agent to form a mixture, forming a foam from the mixture, and
drying the foam to form a hemostatic sponge.

73. The method of claim 72, wherein the hemostatic sponge consists essentially of acidified gelatin crosslinked to chitosan via a crosslinking agent.

74. The method of claim 72, wherein the w/w ratio of gelatin to chitosan in the aqueous solution ranges from about 4:1 to 1:1.

75. The method of claim 72, wherein the crosslinking agent is an aldehyde in an amount ranging from 0.01 to 0.2% v/v.

76. The method of claim 72, wherein forming the foam comprises vigorous mixing of the mixture at 500 to 4,000 rpm for 1 to 30 mins while reducing the temperature of the mixture to 5-18° C.

77. A hemostatic sponge comprising acidified gelatin crosslinked to chitosan via a crosslinking agent in the form of a foam, sponge, or in particulate or powder form.

78. The hemostatic sponge of claim 77, wherein the gelatin is bovine gelatin or has the same amino acid sequence or content as bovine gelatin.

79. The hemostatic sponge of claim 77, wherein the gelatin comprises human gelatin or has the same amino acid sequence or content as human gelatin.

80. The hemostatic sponge of claim 77, wherein the sponge is made by
contacting acidified gelatin and chitosan to form an aqueous solution in the presence of a crosslinking agent to form a mixture,
forming a foam from the mixture, and
drying the foam to form a hemostatic sponge.

81. The hemostatic sponge of claim 77, further comprising drying the foam to produce the sponge in the form of a sheet.

82. The hemostatic sponge of claim 77, wherein the foam is produced without introduction of a surfactant.

83. The hemostatic sponge of claim 77 that was produced by desiccating the sponge without freezing and converting the sponge to sponge particles or to a sponge powder.

84. A method of treatment comprising contacting a wound or site of bleeding of a subject with the hemostatic sponge of claim 77.

85. The method of claim 84, wherein the subject is human.

86. The method of claim 84, wherein the subject is a veterinary subject.

87. The method of claim 84, wherein the sponge is administered as a particle or powder.

88. The method of claim 84, wherein the wound is an epidermal wound.

89. The method of claim 84, wherein the wound or site of bleeding is in the mouth.

90. The method of claim 84, wherein the would or site of bleeding is in the gastrointestinal tract or to an internal organ.

91. The method of claim 84, wherein the found is a gunshot, blast, shrapnel, or other combat wound.
